(19) United States
(12) Patent Application Publication
(10) Pub. No.: US 2023/0019545 A1
(43) Pub. Date: Jan. 19, 2023

(54) AN APPARATUS

(71) Applicant: Oribiotech LTD, London, Greater London (GB)

(72) Inventors: Farlan Veraitch, London, Greater London (GB); William Raimes, London, Greater London (GB); Richard Smith, Cambridgeshire (GB); Neil Litten, Reading, Berkshire (GB); Michael Sawford, Northamptonshire (GB)

(21) Appl. No.: 17/757,663

(22) PCT Filed: Dec. 6, 2020
(86) PCT No.: PCT/GB2020/053231

§ 371 Date: Jun. 17, 2022

(30) Foreign Application Priority Data
Dec. 17, 2019 (GB) 1918665.9

(51) Int. Cl. B01L 3/00

(52) U.S. Cl. CPC B01L 3/502715; B01L 2200/026; B01L 2300/046; B01L 2200/0689

(57) ABSTRACT

An apparatus suitable for handling biological material, for directly introducing or removing material to, or from, a container, comprising a body comprising at least one resealable port; a cover comprising an aperture and wherein the cover is disposed over an upper side of the body, and wherein the cover is moveable such that an aperture of the cover is configured to expose at least part of the resealable port.

### CROSS-REFERENCE TO RELATED APPLICATIONS

This application is a national phase entry under 35 U.S.C. § 371 of International Patent Application PCT/GB2020/053231, filed Dec. 16, 2020, designating the United States of America and published as International Patent Publication WO 2021/123762 A1 on Jun. 24, 2021, which claims the benefit under Article 8 of the Patent Cooperation Treaty to United Kingdom Patent Application Serial No. 1918665.9, filed Dec. 17, 2019.

### TECHNICAL FIELD

The disclosure relates to an apparatus for directly introducing or removing material to, or from, a container. The apparatus described herein is suitable for handling biological material, particularly for use in one or more unit operations in cell processing, such as in cell and/or gene therapy manufacture. Additionally, the disclosure relates to systems and methods making use of the apparatus described herein.

### BACKGROUND

Cell and gene therapy (CGT) manufacturing processes are often complex and include manual or semi-automated steps across several devices. Equipment systems used in various steps or unit operations, of cell-based therapeutic products (CTP) manufacturing may include devices for various unit operations. The unit operations may include, for example, cell collection, cell isolation, selection, cell expansion, cell washing, volume reduction, cell storage or transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example, but not limited to, stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example, in an appropriate culture medium when culturing the cells. The known devices include shaker flasks, roller bottles, T-flasks and bags. Such bottles or flasks are widely used but suffer from several drawbacks. Chief among the problems are the requirement for transfer of cells without contamination when passaging or processing.

Additionally, the sterile addition of supplements and factors, is difficult and time consuming. The existing cell culture devices require re-supply of culture medium and oxygen for continued cell growth. Such gas permeable cell culture devices are generally known. However, such devices also require transfer of medium and/or cells in and out of the devices.

Collapsible cell processing devices for use in medicine are also known. However, such devices are not fabricated or constructed for use in cell or gene therapy manufacturing unit operations (i.e., steps).

A current problem in the production of cells or gene therapies for use in medicine is the absence of compact, automated closed systems for performing unit operations without contamination. For example, during cell culture, upstream or subsequent processing of cells, there is a risk of contamination when making additions to the culture vessel, or when removing cells or removing liquid samples. The current operating systems are largely manual and hence expensive to operate. Multiple pieces of equipment are typically required to cover all of the non-cell culture steps, which involves many transfers, each of which is an opportunity for operator errors and contamination to occur.

Furthermore, with increasing manual operations comes increasing risk of manual errors and therefore the current labor-intensive processes may lack the robustness required for the manufacture of clinical-grade therapeutics.

Therefore, there is a need for cell processing devices (e.g., multistep cell processors) that permit such processing but reduces, or avoids, the requirement for constant movement of cells into fresh devices. For example, it would be advantageous if scale-up of cells in culture could be achieved without transfer of cells into a larger device as the cell population for any given culture increases. Furthermore, it would be advantageous if cell processing devices could be less reliant on manual operation, as known in previous cell manufacturing devices, which use complex equipment that is large and difficult to assemble and/or operate. The devices use complex networks of tubing, valves and pumps to link elements of the equipment together.

Therefore, it is an object of the present disclosure to provide improved cell and/or gene therapy processing equipment, specifically an apparatus that allows for the introduction and removal of material to or from a container. It is desirable that the apparatus combines the advantages of the cell culture containers that avoid the need for pumps and the requirement for constant passaging of cells into fresh culture devices, holding vessels, tubes etc., with the advantages conferred by having individually configurable cell and/or gene therapy processing devices. Together with a processing unit or system as described herein, the apparatus described herein permits a variety of unit processes to be performed within a single device or container having a smaller footprint and being less complex than existing equipment, as will be explained in more detail herein. Furthermore, the apparatus described herein allows for greater compatibility with automated systems.

Moreover, a cell culture container in which the wall element, being composed of a flexible material, is compressible with respect to its top and base sections, is compatible with the apparatus described herein. Cell culture containers having at least one inlet and further comprising one or more auxiliary containers in fluid communication with the primary container is also compatible with the apparatus described herein.

### BRIEF SUMMARY

According to one aspect of the disclosure, there is provided an apparatus, for directly introducing or removing material to, or from, a container, comprising:
a body comprising at least one resealable port;
a cover comprising an aperture and wherein the cover is disposed over an upper side of the body, and wherein the cover is moveable, the aperture of the cover is configured to expose at least part of the resealable port when the aperture is disposed over the resealable port.

That is, the apparatus includes a first component, a body, and a second component, a cover. The body may be formed as a disc, a plate, a wall or the like, and serves to house the at least one resealable port. In some embodiments, the body surrounds the at least one resealable port. Ideally, the body encompasses, or houses, the at least one resealable port, enough to hold the at least one resealable port securely. The cover serves to cover, i.e., overlay, at least a portion of one or more sides or faces of the body.

The at least one resealable port serves to provide passageway, of material, for example, a fluid or solid, upon engagement with an appropriate mechanism, for example, a hollow needle. The at least one resealable port is resealable in the sense that it is resealable or otherwise substantially returns to its original configuration after use. There may be one, one or more, or a plurality of resealable ports.

The cover includes an aperture that may expose, present or otherwise makes available to the atmosphere, at least part of the resealable port. The aperture of the cover may be configured to expose some, most, or all of the resealable port. There may be more than one aperture. In some embodiments, the cover comprises a plurality of apertures. The aperture of the cover may expose a single resealable port. The aperture of the cover may expose a plurality of resealable ports.

The aperture of the cover may extend in its entirety to expose the entirety of the resealable port of the body when the aperture is disposed over the resealable port. In some embodiments, the aperture of the cover is configured to expose the entirety of the resealable port.

The aperture of the cover may, or may not be covered, in use, by a portion of the cover, for example, a door, a diaphragm, or the like. Such a door, diaphragm, or the like, may be engageable, moveable or the like to uncover the aperture and expose at least part of the resealable port. In some embodiments, the cover comprises a door. In some embodiments, the door covers the aperture of the cover. In some embodiments, the door is moveable between an open and a closed position. In some embodiments, the door is moveable between an open and a closed position wherein, in the closed position, the door covers the aperture of the cover. In specific embodiments, the door of the cover comprises a transparent portion or window. This may enable the user to know if the resealable port of the body is located adjacent to, or near to, the aperture of the cover without needing to open the door of the cover. In other embodiments, the door may be biased to a closed position. In some examples, the bias may be a spring bias. In some embodiments, the biasing mechanism of the door of the cover comprises a spring.

The present disclosure provides the advantage that the apparatus described herein is less complex in construction and is more suited for automated and multi-step processes, or, at the very least, multi-step processes requiring less user interaction and/or intervention. Thus, the apparatus described herein allows for one or more unit operations in a cell processing methodology that is less reliant on manual interaction, and thus less prone to manual, i.e., user, errors. Furthermore, the apparatus described herein may be cheaper to manufacture and maintain.

The apparatuses, systems and methods described herein is generally suitable for, but not necessarily limited to, biological and/or chemical processing. Particularly, the apparatuses, system and methods described herein are generally suitable for handling biological material, which requires special technical considerations, such as ensuring an aseptic environment and avoiding sheer, or other stress, to living material. The apparatuses, systems and methods described herein may be used for directly introducing biological material, for example, cellular material, to, or from, a container, such as a bioreactor. More particularly, the apparatus described herein is generally suitable for one or more unit operations in cell processing, such as in cell and/or gene therapy manufacture.

In certain embodiments, the resealable ports may be planar with the body.

In certain embodiments, the body comprises an upper surface and a lower surface, the cover is disposed over the upper surface, and the at least one resealable port extending through the body from the upper surface to the lower surface.

In certain embodiments, the body comprises a plurality of longitudinal passageways extending from the upper surface to a recess formed in the lower surface, each longitudinal passageway comprising a resealable port and the annular recess comprising an annular resealable port.

In certain embodiments, each resealable port in each longitudinal passageway and the annular resealable port are integrally formed. That is, the resealable portion of the body may be formed by one or more longitudinally extending resealable portions terminating in an annular resealable portion.

This provides the advantage that the resealable port cannot be pushed through the body, and thus out of position, in use. Thus, a more secure resealable port arrangement is provided. Moreover, an aseptic, or sterile, environment may be maintained.

In some embodiments, the body comprises a resealable port that is integrally formed. Advantageously, this may allow easy manufacture.

In certain embodiments, the at least one resealable port is a hermetic seal. This provides the advantage that a fluid tight seal may be maintained.

In certain embodiments, the hermetic seal is a septum seal. This provides the advantage that an aseptic, or sterile, environment may be maintained.

In some embodiments, the resealable port is a self-sealing seal. The advantage of this is that the seal of the resealable port can close again automatically after the object causing a pierce is removed. The self-sealing seal is particularly advantageous under circumstances requiring multiple piercing of the seal, i.e., multiple accesses through the apparatus.

In specific embodiments, the resealable port comprises a self-sealing hermetic seal.

In specific embodiments, the resealable port comprises a self-sealing septum seal.

In specific embodiments, the resealable port comprises a self-sealing, hermetic, septum seal.

This provides the advantage that an aseptic, or sterile, environment may be maintained whilst allowing for aseptic, or sterile, disconnection and reconnection due to the resealable properties of septum seals.

In specific embodiments, the resealable port comprises a silicone material or a thermoplastic elastomer material.

In certain embodiments, the at least one septum seal and the body are co-molded.

This provides the advantage that the apparatus is more robust, and thus less susceptible to damage causing immediate failure.

In certain embodiments, the cover further comprising a connector mechanism for connecting to a portion of a container, a bioreactor or a connector.

In certain embodiments, the connector mechanism partially surrounds at least a portion of the aperture of the cover.

This provides the advantage that the user can visually identify which part of the at least one resealable port is in use.

In certain embodiments, the cover comprises a plurality of connector mechanisms, each connector mechanism for connecting to a portion of a container, a bioreactor or a connector. In some examples, each connector mechanism of the plurality of connector mechanisms may partially surround at least a portion of an aperture, of a plurality of apertures, of the cover. Thus, each connector mechanism may partially surround at least a portion of the at least one resealable port. Thus, the cover may comprise a plurality of connector mechanism each connector mechanism partially surrounding at least a portion of an aperture of the cover. In this way, several accesses to the at least one resealable port is provided. Thus, more than one resealable port can be used at the same time. When the cover comprises a plurality of apertures in the cover, some, all or none of the apertures may comprise a door. In some examples, the connector mechanisms may be spaced apart from one another in any appropriate arrangement. In some examples, a first connector mechanism may be diametrically opposite to a second connector mechanism. In some embodiments, the cover comprises an aperture and connector mechanism equal is number to the number of resealable ports of the body.

This provides the advantage that the user can introduce or remove several materials to or from the container. Additionally, this provides the advantage that a user can sample the contents of the container without having to remove other connected elements.

In certain embodiments, the connector mechanism comprises a threaded portion configured to engage with a corresponding threaded portion of a container, a bioreactor or a connector.

In certain embodiments, the connector mechanism comprises a wall wherein the wall is upstanding from the cover, and at least partial surrounds the aperture of the cover. In certain embodiments, the wall of the connector mechanism further comprises circumferential protrusions on the outer surface of the wall of the connector mechanism. In some embodiments, the protrusions of the wall of the connector mechanism are configured and arranged to be able to mate with the corresponding grooves or recesses of a corresponding connector or container, to be connected to the apparatus.

In some embodiments, the connector mechanism is configured and arranged to provide a visual and/or tactile indication upon mating with a corresponding connector or container. In such embodiments, the protrusions of the wall of the connector mechanism may provide a tactile indication, such as a snap-fit engagement, with the corresponding grooves or recesses of the corresponding connector or container.

In certain embodiments, the cover comprises a centrally disposed opening at least partly surrounded by an upstanding wall.

In certain embodiments, the body comprises a central hub having a coupling element, the central hub extending through the centrally disposed opening and the coupling element operably coupled to the upstanding wall.

In certain embodiments, the body is configured to provide fluid communication between an interior volume of a container, to be connected to the apparatus, and an exterior volume of the container. In specific embodiments, the centrally disposed opening and/or the central hub are configured to provide fluid communication between an interior volume of a container, to be connected to the apparatus, and an external volume of the container. The external volume of the container may be the external atmosphere or environment. In particular embodiments, the centrally disposed opening and/or the central hub comprise a filter. The filter may be a gas-permeable liquid-impermeable material. The filter may have a pore size of less than 10 microns, less than 5 microns, less than 1 micron or approximately 0.2 microns.

In some embodiments, the body is configured to provide fluid communication between an interior volume of a first container, to be connected to the apparatus, and an interior volume of a second container, to be connected to the apparatus. In specific embodiments, the centrally disposed opening and/or the central hub are configured to provide fluid communication between an interior volume of a first container, to be connected to the apparatus, and an interior volume of a second container, to be connected to the apparatus. The first container may be a bioreactor and the second container may be a compressible, an expandable, or an inflatable container. The second container may comprise a flexible bag; a container having a top section, a base section and a wall element, wherein the wall element, being composed of a flexible material, is compressible with respect to its top and base sections; a container having a side wall including a plurality of folds therein (i.e., a concertina side wall); or the like. In some embodiments, the second container may comprise gas-permeable liquid-impermeable material.

In some embodiments, the second container may be removably attachable, operably attachable, or the like, to the centrally disposed opening and/or the central hub. In such embodiments, it is preferable that the second container is sealingly couplable to the centrally disposed opening and/or the central hub to maintain an aseptic, or sterile, environment. In some embodiments, the central hub may include, optionally as part of the upstanding wall, a connector element, such as an internally threaded portion, a clip portion, or the like. The internally threaded portion, the clip portion or the like may be configured and arranged to cooperate with a corresponding threaded portion, clip portions, or the like of the second container.

This provides the advantage that a connection to a breathing mechanism may be incorporated within the apparatus.

In certain embodiments, the apparatus further comprises a container operably attached to the connector element. The container may be removably attached, irremovably attached, sealingly engaged, or the like. The container may include a corresponding connector element arranged to cooperate with the connector element of the central hub. In particular embodiments, the connector element and corresponding connector element include complementary threaded portions, such as screw threads, or complementary clip portions. The container may be a breathing container, such as a compressible, an expandable, or an inflatable container, such as those described above.

This provides the advantage that a breathing mechanism, i.e., a container coupled to the centrally disposed opening and/or the central hub, is provided in fluid communication, through the apparatus, with another container, when connected to the apparatus. Thus, changes in fluid volume, such as changes in air volume, of the connected container may be compensated, during use, by the breathing mechanism.

In certain embodiments, the cover comprises a circumferential skirt that is configured and arranged to removably attach to a circumferential rim of the body.

This provides the advantage that the cover may be removable such that the cover and/or the body can be maintained, repaired, cleaned or the like.

In certain embodiments, the cover is clipped to the body.

This provides the advantage that the user can easily remove, or attach, the cover to the body.

In certain embodiments, the cover comprises a circumferential skirt that is configured and arranged to irremovably attach to a circumferential rim of the body.

This provides the advantage that a user cannot disassemble the apparatus, which may be critical to ensuring an aseptic environment.

In some embodiments, the skirt of the cover comprises a circumferential protrusion, or rail, disposed on the inner surface of the skirt. In some embodiments, the body comprises a circumferential groove disposed on the outer surface of the edge of the body. In certain embodiments, the cover comprises a substantially circumferential rail that is configured and arranged to be received within a substantially circumferential groove of the body.

This provides the advantage that the cover is guided within the body, during installation or during use. In some embodiments, the skirt of the cover comprises a groove disposed on the inner surface of the skirt. In some embodiments, the body comprises a protrusion, or rail, disposed on the outer surface of the edge of the body.

The groove and rail arrangement for detachably attaching the cover and body can be alternatively arranged in some embodiments.

In some embodiments, the body and cover are circular in shape. In some embodiments, the body and cover are both generally disc-like in shape each having an upper face and lower face and a peripheral edge.

In certain embodiments, the cover and the body are rotatable with respect to one another.

This provides the advantage that the apparatus is more suited to automated or semi-automated processes.

In certain embodiments, the lower surface of the body includes a connector mechanism for connecting to a portion of a container, a bioreactor or a connector.

In certain embodiments, the connector mechanism may be a threaded mechanism, a clip mechanism, or the like. In certain embodiments, the connector mechanism comprises a threaded portion or a threaded ring, a flange, a threaded flange, a one-way clip, a slide clip or the like.

In other embodiments, the connector mechanism comprises a permanently bonded portion between the container, the bioreactor or the connector and the lower surface of the body. In some examples, the container, the bioreactor or the connector may be welded, for example, thermally welded, or the like, to the lower surface of the body.

In certain embodiments, the lower surface of the body includes a threaded portion configured to engage with a corresponding threaded portion of a container, a bioreactor or a connector.

In certain embodiments, the threaded portion comprises an anti-rotational element configured and arranged to resist rotation of a container, a bioreactor or a connector, in use.

This provides the advantage that the container, bioreactor, connector or the like, in use, do not become detached from the apparatus, particularly where the body is rotated with respect to the cover by virtue of rotating the container, bioreactor, connector or the like.

In certain embodiments, the anti-rotational element comprises one or more trapezoidal threads of the threaded portion.

In certain embodiments, the or each trapezoidal thread forms an inclination angle between a longitudinal axis and a lower surface of the or each trapezoidal thread of between approximately 20 degrees and approximately 80 degrees, preferably between approximately 30 degrees and approximately 50 degrees. The inclination angle may have a lower limit of approximately 20 degrees, approximately 30 degrees, approximately 40 degrees, approximately 50 degrees, approximately 60 degrees or approximately 70 degrees, or any integer therebetween. The inclination angle may have an upper limit of approximately 30 degrees approximately 40 degrees, approximately 50 degrees, approximately 60 degrees, approximately 70 degrees or approximately 80 degrees, or any integer therebetween. Any combination of lower limit and upper limit is contemplated herein.

In certain embodiments, the body and the cover are generally planar and circular in shape, both having an upper and lower face or surface. This advantageously enables easy rotation of the cover in relation to the body.

In certain embodiments, the body further comprises a detachable cover (i.e., an aseptic barrier) disposed over at least a portion of the at least one resealable port. In specific embodiments, the body further comprises a detachable cover disposed over the at least one resealable port. In some embodiments, the cover further comprises a detachable cover disposed over the, or each, resealable port. In some embodiments, the detachable cover of the body comprises an aseptic paper cover. In some embodiments, the aseptic paper cover may comprise a handle.

This provides the advantage that a user can ensure an aseptic, or sterile, environment at the, at least one resealable port.

In certain embodiments, the detachable cover comprises an aseptic barrier, such as an aseptic paper seal, configured to mate, for example, with a corresponding aseptic barrier, such as an aseptic paper seal, of a corresponding connector or container.

The aseptic barrier may include an aseptic membrane arranged to cooperate with a corresponding aseptic membrane. The aseptic barrier may further include a rigid portion, or a rigid housing or a clip portion, operably coupled to the aseptic membrane. The rigid portion, or the rigid housing, may further include an element, such as an aperture, arranged to be operably engaged by a portion of an aseptic barrier removal system.

This provides the advantage that the apparatus is readily compatible with other components, and so is more suited to an automated or multi-step process.

In certain embodiments, the detachable cover comprises a handle.

This provides the advantage that the user can easily remove the detachable cover, in use.

In certain embodiments, the detachable cover is configured such that it is detachable when a container is connected to the connector mechanism of the cover.

This provides the advantage that the user can ensure that a correct connection between the connector mechanism and the container is achieved.

The present disclosure provides a bioreactor comprising an apparatus as described herein.

In another embodiment, there is provided a bioreactor sealingly coupled to an apparatus as described herein.

In some embodiments, the apparatus as described herein is a lid, an interface plate, or a cassette. In specific embodiments, the apparatus as herein described is a lid, for example, of a container. In certain embodiments, the apparatus as described herein is a lid of a bioreactor.

According to another aspect of the disclosure, there is provided a method of performing one or more operations in cell and/or gene therapy manufacture using an apparatus or a system as described herein.

According to another aspect of the disclosure, there is also provided a system comprising:
an apparatus as described herein;
a container, a bioreactor or a connector, or any combination of container, bioreactor or connector, sealingly engaged with the at least one resealable port.

In certain embodiments, the system further comprises:
a housing for enclosing the apparatus and the container, bioreactor or connector, or any combination of container, bioreactor or connector;
a drive mechanism disposed within the housing and configured to rotate the body of the apparatus, in use.

In certain embodiments, the housing encloses an actuating mechanism configured to actuate a portion of the container, bioreactor and/or connector thereby providing a fluid connection through the at least one resealable port.

In certain embodiments, the system comprises a bioreactor sealingly engaged with the at least one resealable port at the lower surface of the body, and at least one container sealingly engaged with the at least one resealable port at the upper surface of the body.

In certain embodiments, the system comprises at least one connector having a distal end and a proximal end, the proximal end of the connector sealingly engaged with the at least one resealable port at the upper surface of the body and the distal end of the connector sealingly engaged with the container.

In certain embodiments, the at least one resealable port comprises a hermetic seal.

In certain embodiments, the hermetic seal comprises a septum seal.

In certain embodiments, the at least one resealable port comprises a self-sealing seal. In certain embodiments, the, some of, or each, resealable port comprises a self-sealing seal. In specific embodiments, the, some of, or each, resealable port comprises a self-sealing septum seal.

According to another aspect of the disclosure, there is also provided a method of operating an apparatus for directly introducing or removing material to, or from, a container, comprising:
providing an apparatus as described herein;
sealingly engaging a first container to the at least one resealable port at the lower surface of the body;
sealingly engaging a second container to the at least one resealable port at the upper surface of the body;
providing fluid communication between the container and the bioreactor by providing a fluid passageway through the at least one resealable port;
introducing material from the second container into the first container, or removing material from the first container into the second container.

In certain embodiments, the step of sealingly engaging a container to at least one resealable port at the upper surface of the body further comprises:
providing a connector;
sealingly engaging a proximal end of the connector to the at least one resealable port at the upper surface of the body; and
sealingly engaging the container to a distal end of the connector.

In certain embodiments, the step of providing fluid communication between the container and the bioreactor further comprises piercing the at least one resealable port with a hollow needle.

In certain embodiments, the resealable port comprises a hermetic seal.

In certain embodiments, the hermetic seal comprises a septum seal.

As used herein, the term “aseptically attached” is used to describe an attachment that, when attached, prevents the passage of microbes.

As used herein, the term “body” is used to describe an element or wall.

As used herein, the term “container” is used to describe any vessel capable of holding a material, for example, a fluid, and includes both hard and soft containers, for example, sacks, bags, bioreactors, bellows and vacutainers.

As used herein, the term “door” is used to describe a moveable element that can be moved between an open and closed position such that when the door is in a closed position, the aperture is covered by the door and that when the door is in an open position at least a portion of the aperture is not covered.

As used herein, the term “material” is used to describe solids and fluids, that is, liquids and gases. The term “material” also encompasses gels, pastes, suspensions of solids in solution, or the like.

As used herein, the term “fluid” is used to describe gases and liquids, including solutions, but also includes granular solids including powders. The granular solids need not be in solution. Equally, the granular solids may be in a solution, for example, the granular solids may be suspended within a liquid.

As used herein, the term “hermetic seal” is used to describe a seal that is fluid tight.

As used herein, the term “to pierce” is used to describe the piercing of a material, such that the piercing element, for example, a hollow needle, at least partially protrudes through the material.

As used herein, the term “resealable” is used to describe a seal that is able to reseal, or close again after the seal has been pierced, and the object causing the pierce, for example, a hollow needle, has been removed.

As used herein, the term “self-sealing” is used to describe a seal that is able to reseal without manual intervention, or close again after the seal has been pierced, and the object causing the pierce, for example, a hollow needle, has been removed.

As used herein, the term “sealingly engaged” is used to describe an engagement, or connection that seals. The term “sealingly engage” used herein includes a fluid tight seal.

As used herein, the term “septum seal” is used to describe a seal that comprises a material to give an aseptic seal. This term may include preventing or reducing the passage of microbes.

Any of the features or steps described herein in relation to one embodiment, aspect or example, or of the method for manufacturing, the apparatus (or system) of manufacturing a component for or thereof, or a kit of parts comprising an apparatus or the manufacturing apparatuses thereof of the method for manufacturing an apparatus, any of the apparatus (including the system) for manufacturing a component for an apparatus, a kit of parts comprising a plurality of manufacturing apparatuses suitable for the manufacturing of an apparatus as described herein, or a component for an apparatus thereof, may be equally applicable to any other embodiment, aspect or example of any apparatus herein described, any of the method of manufacturing a component for an apparatus as herein described, an apparatus (including the system) for manufacturing a component of an apparatus, a component thereof, or a kit of parts comprising a plurality of manufacturing apparatuses suitable for the manufacturing of an apparatus.

Reference will now be made to the drawings, which depict one or more embodiments described in this disclosure. However, it will be understood that other embodiments not depicted in the drawings fall within the scope of this disclosure. Like numbers used in the figures refer to like components, steps and the like. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components. The figures are presented for purposes of illustration and not limitation. Schematic drawings presented in the figures are not necessarily to scale.

### BRIEF DESCRIPTION OF THE DRAWINGS

These and other aspects, features and advantages of which embodiments of the disclosure are capable of, will be apparent and elucidated from the following description of example embodiments and aspects of the present disclosure, reference being made to the accompanying drawings, in which:

FIG. 1 illustrates a perspective view of a body of the apparatus according to the present disclosure;

FIG. 2 illustrates the body of FIG. 1 from below;

FIG. 3 illustrates the body of FIG. 1 having a plurality of aseptic paper seals attached;

FIG. 4 illustrates a perspective view of a cover of the apparatus according to the present disclosure;

FIG. 5 illustrates the cover of FIG. 4 from below;

FIG. 6 illustrates a perspective view of a system, including the apparatus according to the present disclosure, and an aseptic connector and a container attached thereto;

FIG. 7 illustrates a cross-sectional view of the apparatus according to the present disclosure, including a container attached thereto;

FIG. 8 illustrates an enlarged view of FIG. 7;

FIG. 9 illustrates a cross-sectional view of the system as shown in FIG. 6;

FIG. 10 illustrates a container for use with the apparatus according to the present disclosure;

FIG. 11a illustrates a perspective view of a system having the apparatus according to the present disclosure and a container attached thereto, and an aseptic connector before a connection is made to the apparatus, and FIG. 11b illustrates a top view of FIG. 11a;

FIG. 12a illustrates a perspective view of the system of FIGS. 11a and 11b having an aseptic connector attached to the apparatus, and FIG. 12b illustrates a top view of FIG. 12a;

FIG. 13a illustrates a perspective view of the system of FIGS. 12a and 12b having aseptic paper seals removed, and FIG. 13b illustrates a top view of FIG. 13a;

FIG. 14a illustrates a perspective view of the system of FIGS. 13a and 13b once the aseptic connector has been removed from the apparatus, and FIG. 14b illustrates a top view of FIG. 14a;

FIG. 15a illustrates a perspective view of the system of FIGS. 11a to 14b after use, and FIG. 15b illustrates a top view of FIG. 15a;

FIG. 16a illustrates a perspective view of the system of FIGS. 15a and 15b once rotated in the direction indicated, and FIG. 16b illustrates a top view of FIG. 16a;

FIG. 17a illustrates a perspective view of the system of FIGS. 16a and 16b, including the apparatus according to the present disclosure and a container attached thereto, and an aseptic connector before a connection is made to the apparatus, and FIG. 17b illustrates a top view of FIG. 17a;

FIG. 18a illustrates a perspective view of the system of FIGS. 17a and 17b having an aseptic connector attached to the apparatus, and FIG. 18b illustrates a top view of FIG. 18a;

FIG. 19a illustrates a perspective view of the system of FIGS. 18a and 18b having aseptic paper seals removed, and FIG. 19b illustrates a top view of FIG. 19a;

FIG. 20 illustrates a perspective view of a system according to the present disclosure with an apparatus partially loaded therein;

FIG. 21 illustrates a perspective view of a mounting bracket, actuators and frictional drive mechanism of the system of FIG. 20; and

FIG. 22 illustrates a top view of the mounting plate and the frictional drive mechanism of the system of FIG. 21.

### DETAILED DESCRIPTION

The described example embodiments relate to an apparatus for directly introducing or removing material to or from a container. Other embodiments relate to a system and a method for introducing or removing material to or from a container, either manually or in a semi-automated or an automated manner. Further embodiments, examples, aspects and advantages will become apparent through the remaining description.

Certain terminology is used in the following description for convenience only and is not limiting. The words “upper” and “lower” designate directions in the drawings to which reference is made and are with respect to the described component when assembled and mounted. The words “inner,” “inwardly,” “outer,” and “outwardly” refer to directions toward and away from, respectively, a designated centerline or a geometric center of an element being described (e.g., a central axis), the particular meaning being readily apparent from the context of the description. Further, the terms “proximal” (i.e., nearer to) and “distal” (i.e., away from) designate positions relative to an axis or a point of attachment.

Further, as used herein, the terms “connected,” “affixed,” “coupled” and the like are intended to include direct connections between two members without any other members interposed therebetween, as well as, indirect connections between members in which one or more other members are interposed therebetween. The terminology includes the words specifically mentioned above, derivatives thereof, and words of similar import.

Further, unless otherwise specified, the use of ordinal adjectives, such as, “first,” “second,” “third,” etc., merely indicate that different instances of like objects are being referred to and are not intended to imply that the objects so described must be in a given sequence, either temporally, spatially, in ranking or in any other manner. Like reference numerals are used to depict like features throughout.

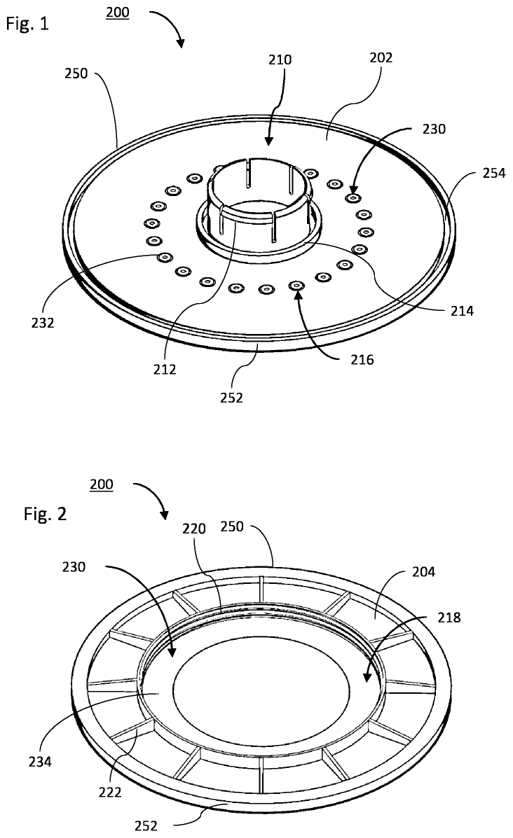

As best shown in FIGS. 1 to 9, there is provided an apparatus 100 including a body 200 and a cover 300. The body 200 or the cover 300 or both the body 200 and the cover 300 may be moveable with respect to one another. In the depicted example, the cover 300 is slidably mountable to the body 200 such that the body 200 or the cover 300 may be rotated with respect to one another.

Referring more specifically to FIGS. 1 to 3, the body 200 includes an upper surface 202 and a lower surface 204 and is generally circular and planar. The body 200 further includes a central hub 210 upstanding from the upper surface 202 of the body 200 along a central longitudinal axis. The central hub 210 includes a coupling element, formed as a plurality of hooks 212, at a distal end thereof. Each hook of the plurality of hooks 212 are configured to engage with a corresponding portion of the cover. Specifically, the hooks 212 in the depicted embodiment are clipped, via a snap-engagement mechanism, to an upstanding wall of the cover, as described further below. The central hub 210 may also include an annular ring 214 surrounding and enclosing the central hub 210, the annular ring 214 being coaxial with the central hub 210 and extending from the upper surface 202. Additionally, although not shown in the present example, the central hub 210 includes a further coupling element, formed interiorly of the central hub 210, configured and arranged to allow coupling of the central hub 210, and thus the body 200 and apparatus 100, to a container, which generally forms part of a breathing mechanism. Such further coupling element is provided by screw threads or a clip that is configured and arranged to cooperate with corresponding screw threads or a clip of the container.

The upper surface 202 of the body 200 includes a plurality of longitudinal passageways 216 extending from the upper surface 202 longitudinally into the body 200. The lower surface 204 of the body 200 includes a generally annular recess 218 having screw threads 220. The lower surface 204 of the body 200 also includes a plurality of radial ribs 222 extending from the annular recess 218 towards an outer edge of the body 200. Each of the plurality of longitudinal passageways 216 extend from the upper surface 202 to the annular recess 218 on the lower surface 204.

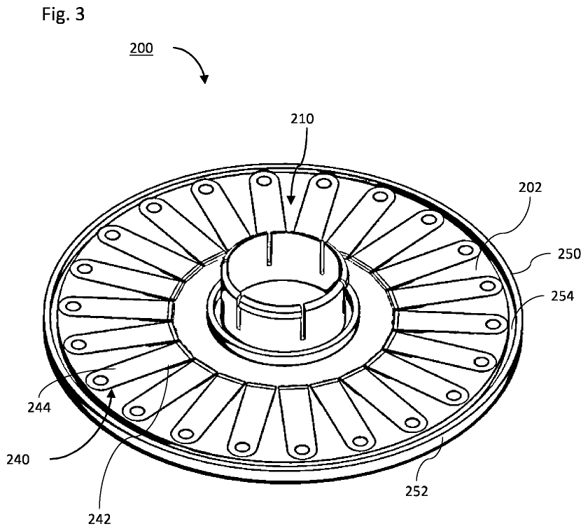

The screw threads 220 are configured and arranged to receive a corresponding threaded portion of a corresponding container. The screw threads 220, as best shown in FIGS. 2 and 8, include an anti-rotational element, formed as a trapezoidal thread 221 as best shown in FIG. 8, configured and arranged to resist rotation of an attached container. The trapezoidal thread 221 includes an upper thread surface, a side surface and a lower thread surface. The lower thread surface forms an inclination angle with the longitudinal axis of between approximately 20 degrees and approximately 80 degrees. In some examples, the inclination angle may be between approximately 30 degrees and approximately 50 degrees. In other examples, the inclination angle may have a lower limit of approximately 20, 30, 40, 50, 60 or 70 degrees, or any integer therebetween, and an upper limit of approximately 30, 40, 50, 60, 70 or 80 degrees, or any integer therebetween. As will be appreciated by the person skilled in the art, other anti-rotational elements may also be used.

The body 200 also includes a plurality of resealable ports, formed as a septum seal member 230 in the depicted embodiment. In this embodiment, the septum seal member 230 includes, through the resealable ports, a plurality of protruding upper portions 232 facing the cover direction. The septum seal member 230 comprises protruding upper portions 232 extending longitudinally from an upper surface of a generally annular portion 234 of the septum seal member 230. That is, the septum seal member 230 has an integrally formed plurality of protruding upper portions 232 and a generally annular portion 234.

Additionally, the body 200 includes a plurality of aseptic paper seals 240, as best shown in FIG. 3, each aseptic paper seal 240 disposed over the uppermost, exposed, surface of each of the protruding upper portion 232 of the septum seal member 230. Each aseptic paper seal 240 includes an aseptic membrane 242 and a handle 244 extending therefrom. Each aseptic paper seal 240 serves to maintain an aseptic uppermost surface of each of the protruding upper portion 232 and thus the upper surface portion of the septum seal member 230.

The body 200 also includes a circumferential skirt 250 at the outermost edge 252 of the body 200, the outermost edge 252 extending between the upper surface 202 and the lower surface 204. The circumferential skirt 250 includes one or more circumferential grooves 254 for receiving a portion of the cover 300, as described below.

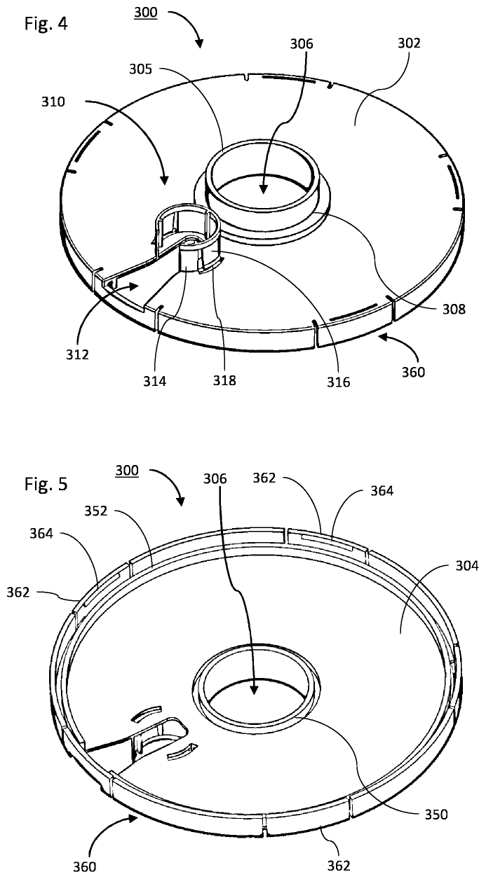

Referring now to FIGS. 4 and 5, the cover 300 includes an upper surface 302 and a lower surface 304. The upper surface 302 includes an upstanding wall 305 enclosing a centrally disposed opening 306. The upstanding wall 305 is surrounded by a circumferential flange 308. The upper surface 302 also includes a connector mechanism 310 for connecting, for example, a container to an upper surface 302 of the cover 300. The connector mechanism 310 at least partially surrounds at least a portion of an aperture 312 formed within the upper surface 302 of the cover 300.

The connector mechanism 310 can be formed in any appropriate way, for example, as a threaded portion or the like. In the described embodiment, the connector mechanism 310 includes a wall 314 upstanding from the upper surface 302 of the cover 300, and a plurality of legs 316 protruding outwardly from the wall 314, each leg 316 having a protrusion 318 at a distal end. The protrusions 318 are configured and arranged to mate with a corresponding groove of, for example, a container, a connector or the like, when connected to the connector mechanism 310.

The lower surface 304 of the cover 300 includes a centrally disposed annular recess 350 enclosing the centrally disposed opening 306. The lower surface 304 of the cover 300 also includes a generally circumferential rail 352 configured and arranged to be received within the substantially circumferential grooves 254 of the body 200, as described below.

The cover 300 also includes a generally circumferential skirt 360 longitudinally extending from the upper surface 302 towards and past the lower surface 304, thereby forming an overhanging portion. The circumferential skirt 360 includes a plurality of cut out portions 362 each having a hook portion 364 at a distal end thereof. The hook portions 364 are configured and arranged to clip to the body 200, as described below.

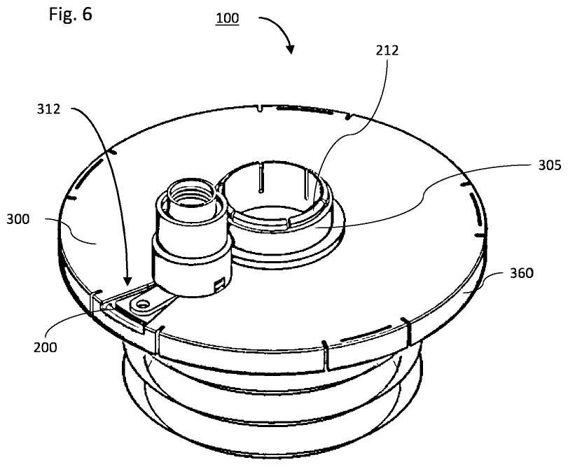

In use, in this embodiment, the cover 300 is secured to the body 200, for example, clipped to the body 200, such that the cover 300 and the body 200 are slidable with respect to one another. In particular, as best shown in FIGS. 6 to 9, the cover 300 is clipped to the body 200 such that the circumferential skirt 360 surrounds the outermost edge 252 of the body 200, and such that the hook portions 364 are clipped, for example, by a snap engagement, with lower surface 204 of the body 200. The circumferential rail 352 of the cover 300 is received within the circumferential groove 254 of the body 200. Thus, the cover 300 and the body 200 are provided to be moveable in a sliding and rotatable manner.

Furthermore, the central hub 210 of the body 200 extends through the centrally disposed opening 306 such that the hooks 212 of the body 200 are clipped, for example, by a snap engagement, with a distal edge of the upstanding wall 305. Additionally, the annular ring 214 of the body 200 is received within the centrally disposed annular recess 350 of the cover 300.

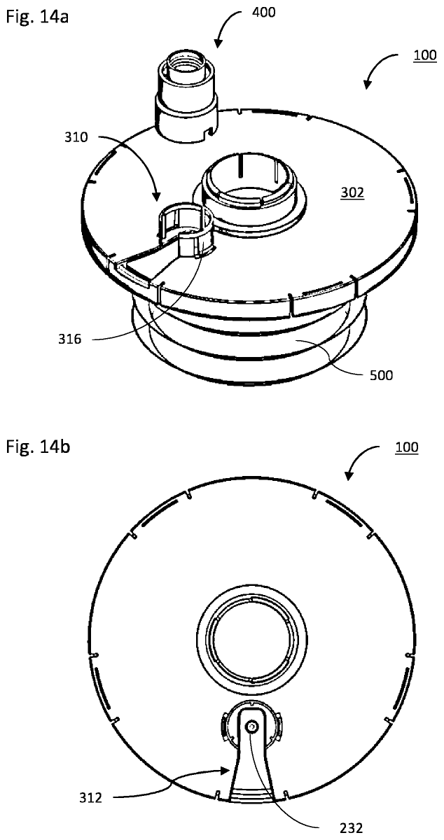

As best shown in FIGS. 6 and 14b, the aperture 312 exposes one of the protruding upper portions 232 of the septum seal member 230 formed within the body 200. Thus, the aperture 312 exposes at least a portion of the at least one resealable port. The remainder of the cover 300 generally covers the remaining, or at least a portion, of the protruding upper portion 232 of the septum seal member 230. Thus, the cover 300 or the body 200 may be rotated, i.e., in a slidable manner, to expose a number of protruding upper portion 232 of the septum seal member 230. Aptly, in this embodiment, the cover is slid or rotated, with the body stationary, to expose through the aperture a desired resealable port. Specifically, in this embodiment, the cover is slid or rotated, with the body stationary, to expose through the aperture a desired protruding upper portion 232 of the septum seal member 230.

In another embodiment, the cover 300 may remain stationary and the body 200 is caused to rotate.

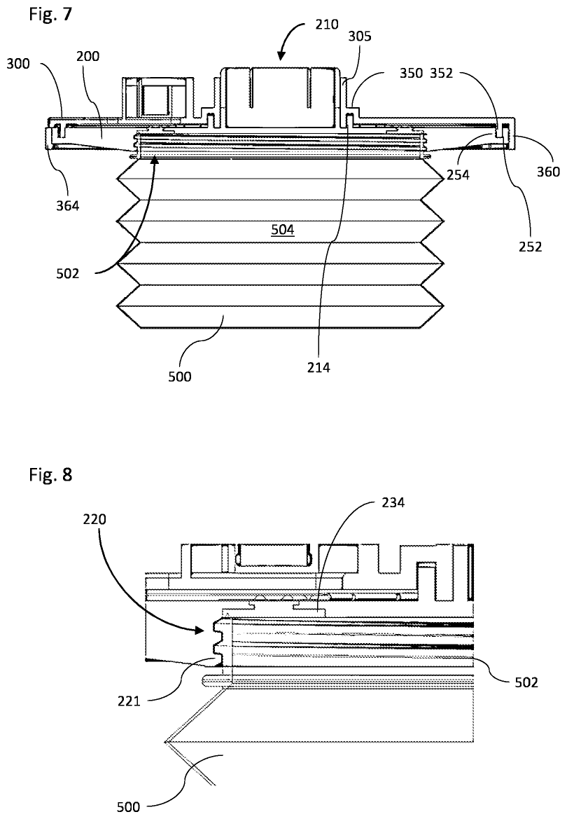

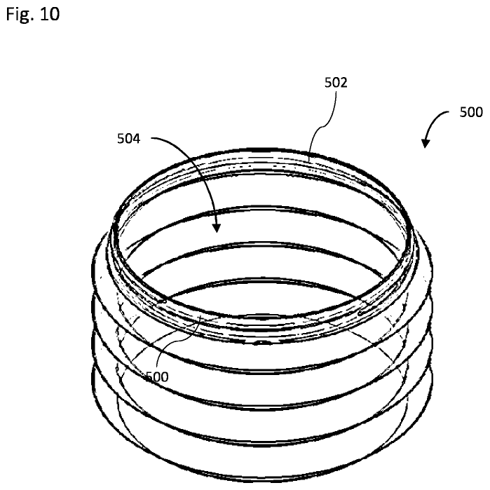

As shown in FIGS. 7 to 10, a container 500, having a thread 502 and a volume of fluid 504, is threadedly engaged with the corresponding screw thread 220 of the body 200. In this way, the container 500 is sealingly engaged at an upper edge thereof, adjacent the thread 502, to the lower surface 204 of the septum seal member 230. The sealing engagement may be a hermetic or liquid seal. The body 200 is then rotated such that one of the protruding upper portion 232 of the septum seal member 230 formed within the body 200 is exposed, when the protruding upper portion 232 of the septum seal member 230 is aligned with the aperture 312. A container or a connector is then engaged with the connector mechanism 310 of the cover 300.

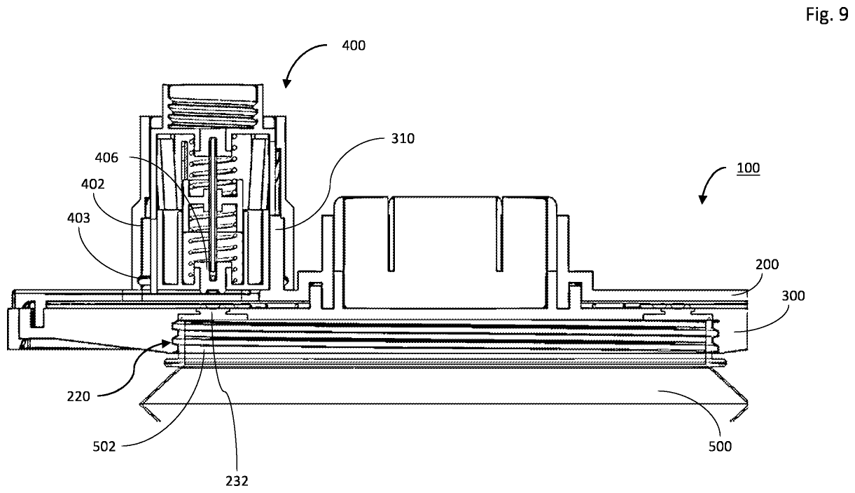

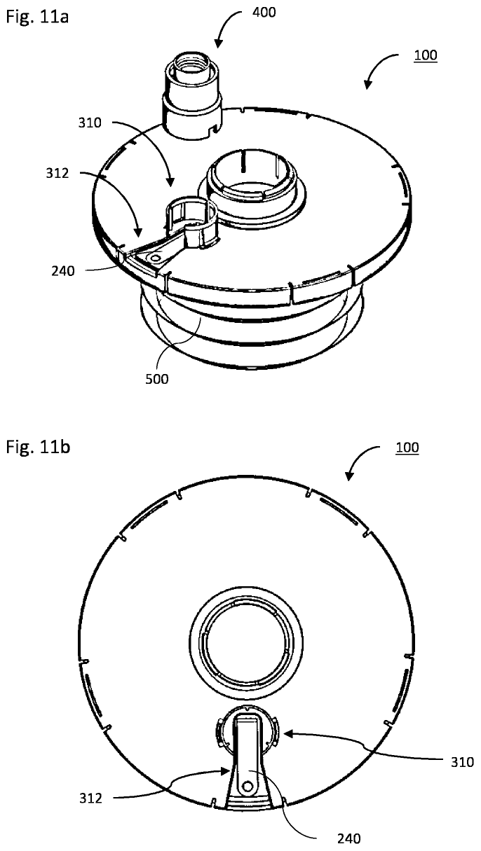

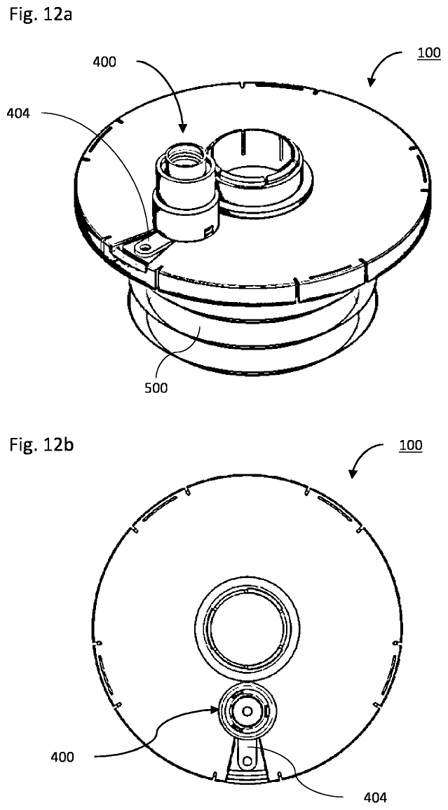

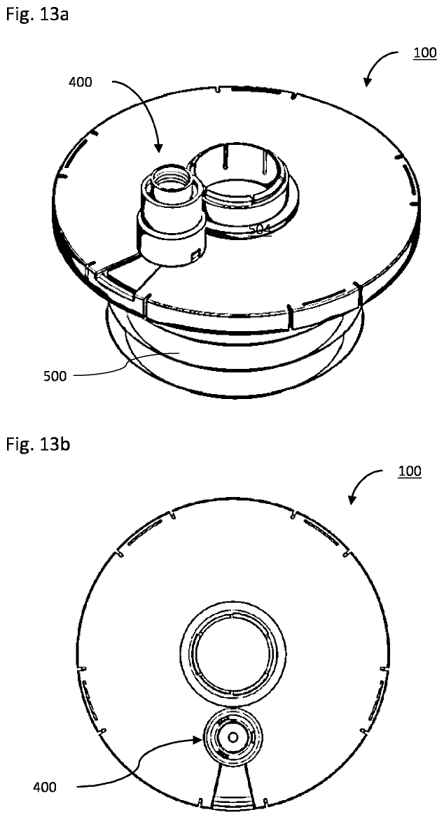

Referring to FIGS. 9 and 11a to 13b, an aseptic connector 400 is connected to the connector mechanism 310. Specifically, an outer sleeve 402 of the aseptic connector 400 includes a circumferential groove 403 that receives the protrusions 318 of each leg 316 of the connector mechanism 310 (see FIG. 4). The aseptic connector 400 also includes an aseptic paper seal 404 (see FIGS. 12a and 12b), the aseptic paper seal 404 comprises a handle and an aseptic membrane. The aseptic paper seal 404 of the aseptic connector 400 is able to align with the aseptic paper seal 240 of the body that is exposed within the aperture 312. The user then removes the aseptic paper seals 240, 404 by pulling the respective handles 244, 406 to allow abutment of the protruding upper portion 232 of the resealable port and a septum seal of the aseptic connector 400.

The user actuates the aseptic connector 400, and/or the container 500, such that a fluid connection between the aseptic connector 400 and the container 500 can be made. For example, in the depicted example, the aseptic connector 400 is actuated such that a hollow needle pierces the seal, thereby providing an aseptic fluid pathway between the aseptic connector 400, and the container 500. In a subsequent step, fluid can be introduced, or removed, from the container attached to the aseptic connector 400 and/or the container 500. The aseptic connector may, for example, be part of a container or be connected to another container.

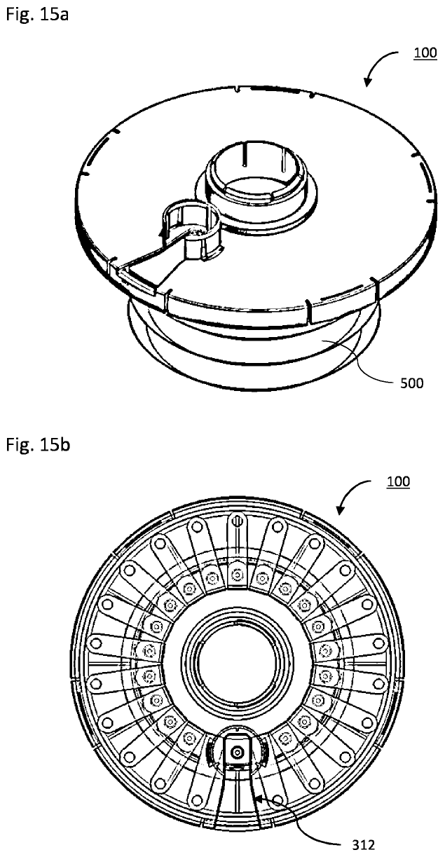

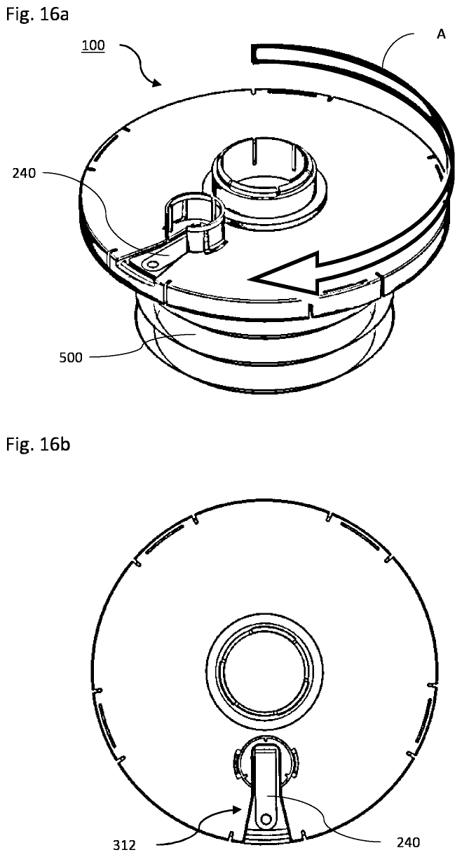

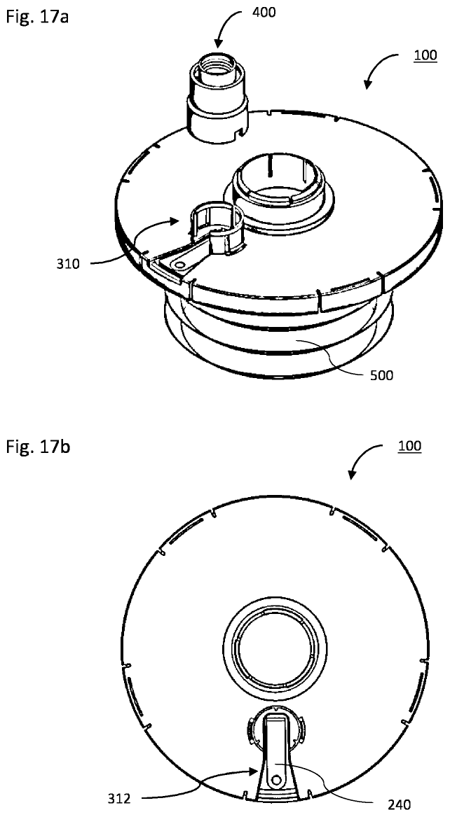

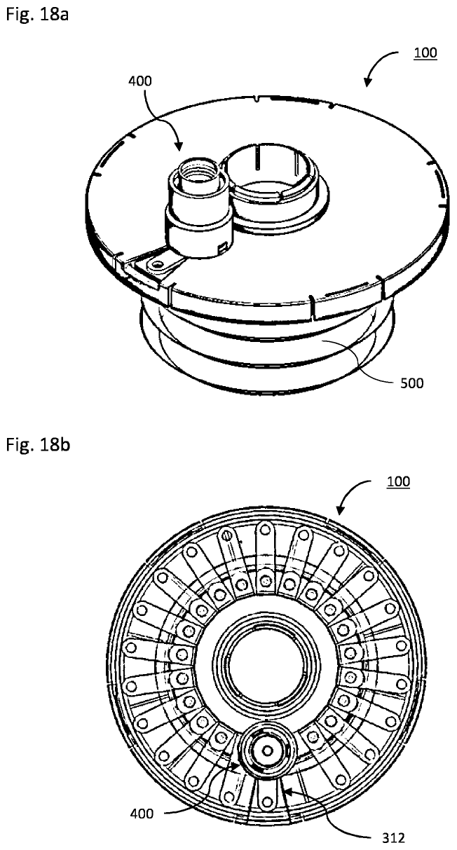

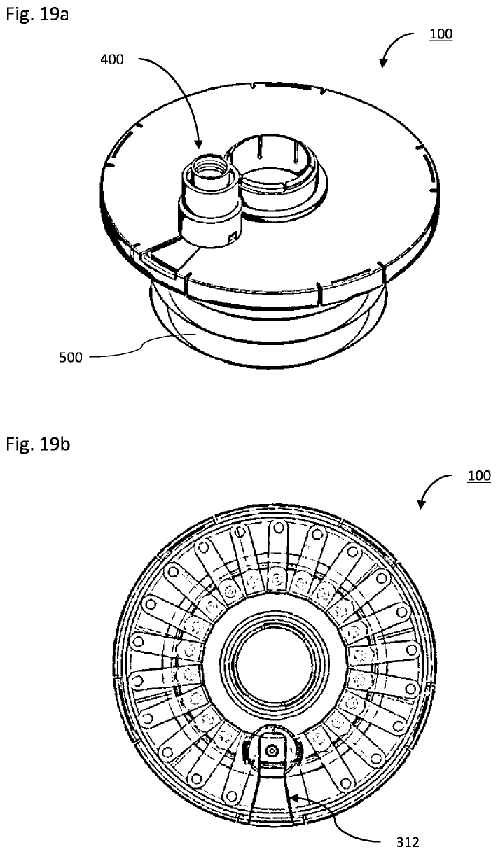

As shown in FIGS. 14a to 16b, the aseptic connector 400 is then removed by pressing the legs 316 of the connector mechanism 310 (see FIG. 4) together and pulling the aseptic connector 400 in a longitudinal direction away from the upper surface 302 of the cover 300. The cover 300 can then be rotated in direction A to expose the next protruding upper portion 232 of the septum seal member 230, covered by the aseptic paper seal 240, in the aperture 312. The process can then be repeated, as shown in FIGS. 17a to 19b.

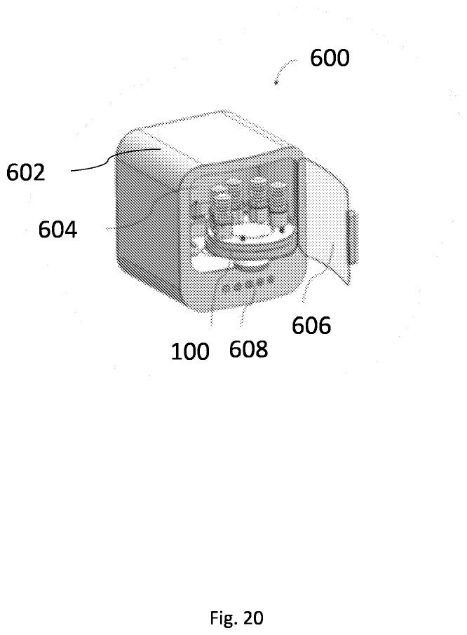

A system 600 is illustrated in FIG. 20, including the apparatus 100 as described herein. The system 600 comprises a housing 602 formed of four walls upstanding from a base wall and a top wall parallel to the base wall and spaced apart from it by the length of the walls. The housing 602 forming a chamber 604 with a hinged door 606 in one wall for receiving the apparatus 100 as described herein. On the front panel of the system 600 is a control panel 608 to enable the user to program and control various features positioned within the chamber 604, as well as their interactions with the apparatus 100. The housing 602, which defines an enclosed space, being chamber 604, houses multiple components in which one or more unit operations (i.e., steps) of cell and/or gene therapy manufacturing process can occur.

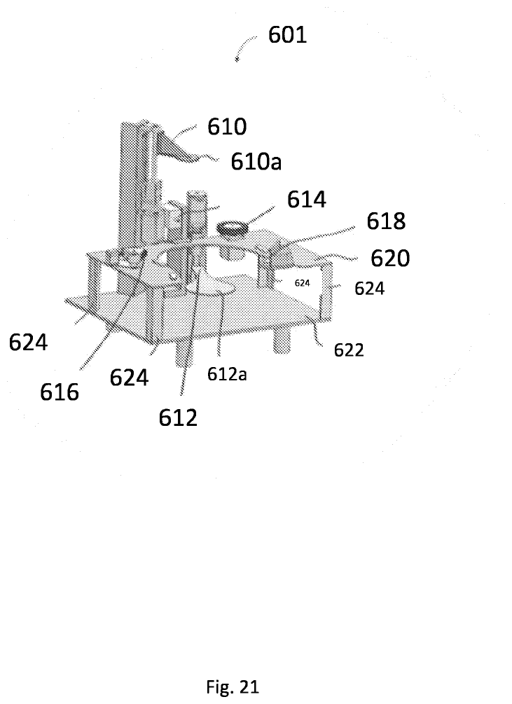

FIG. 21 shows a portion 601 of the system 600 with the housing 602 removed for ease of depiction. Inside the housing 602 the portion 601 of the system 600 comprises a linear actuator 610 for compression of a first container in use, a linear actuator 612 for compression a second container, in use, a friction drive mechanism (614, 616, 618) mounted on a mounting plate 620 and operable to rotate the apparatus 100, or a portion thereof. The internal structure of the apparatus is machined from aluminum, the linear actuators 610, 612 are aluminum and steel constructions with the lead screws hard coated in TFE dry lubricant.

In addition to the mounting plate 620, the mounting bracket comprises a mounting flange (not shown), located above the mounting plate in such a way as to retain the apparatus 100 by frictional fit between the mounting plate 620 and the mounting flange.

The layout of the linear actuators 610, 612 allows them to be hidden in the rear of the apparatus by a cover (not shown) through which only the plungers 610a, 612a protrude to actuate the relevant container and/or connector. In this embodiment, the plungers 610a, 612a may compress bellow-type containers, specifically the bellows of the auxiliary and primary containers respectively. This helps to give a clean and uncomplicated appearance, and provides an apparatus that is simpler to clean and wipe down. A power supply and the electronics for the actuators and the frictional drive mechanism are mounted on the plate 622 below the mounting plate 620. The four risers 624 are adjustable in height and operable to change the distance between the mounting plate 620 and the plate 622 housing the power supply and the electronics. In this way, the apparatus can accommodation different sizes of primary containers.

The housing 602 contains all of the actuators and electronics necessary to manipulate the apparatus 100. The plunger 610a and plunger 612a operable to exert a compression force on the respective containers, and/or a suitable connector, with a maximum force of 100N. The motors driving the linear rails are bipolar stepper motors.

The frictional drive mechanism (614, 616, 618) comprises a drive wheel 614 located on mounting plate 620 and operable to impart rotation on the apparatus 100. The drive wheel 614 is a bipolar stepper motor. The actuator stepper motors on the linear rails and the stepper motor in the frictional drive mechanism are driven by a control system and associated power supply (not shown). The drive wheel 614 may be operable to rotate the body 200, or the cover 300. The drive wheel 614 may engage with a container at the lower surface of the body 200 such that the container and, thus, the body 200, are rotated in use.

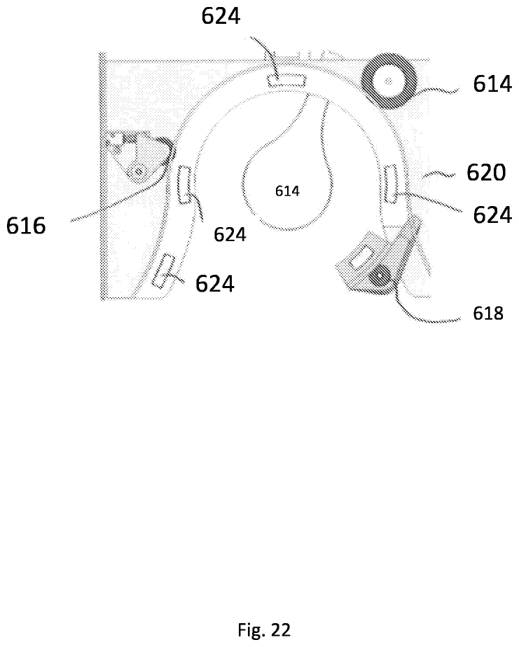

FIG. 22 shows the elements of the frictional drive mechanism (614, 616, 618) mounted to the mounting plate 620 of the mounting bracket. To allow the apparatus 100 and the containers to be inserted from front only, a drive method has been developed where the apparatus 100 may be held between three friction wheels, one of which being driven by drive wheel 614, the other spring loaded 616 and the third being a hinge wheel 618 within the door that opens to allow insertion of the apparatus 100 and closes to lock it in place. The apparatus 100 rotates on low friction PTFE pads 626 on the mounting plate 620. The spring force of the spring-loaded friction wheel 616 will be such that there is no slip between the drive wheel 614 and the outer face of the body 200 or the cover 300 of the apparatus 100. The drive wheel 614 is directly connected to a stepper motor.

The apparatus 100 may also be fitted, as part of the body 200 or the cover 300, with a series of magnets around its circumference so that its position can be read by a Hall Effect sensor mounted on the mounting plate 620. The apparatus 100 therefore acts like an encoder and gives closed loop position feedback independent of any motor slip. The Hall Effect sensor mounted to the mounting plate 620 attached to the housing 602 is operable to detect the magnetic field from the magnets on the apparatus 100 mounted in the housing 602. The Hall Effect sensor is operable to detect the position of apparatus 100 relative to the mounting plate 620.

As will be clear to the person skilled in the art, any appropriate container can be used with the apparatus. For example, in place of an aseptic connector 400 there may be provided a container, a vacutainer or the like, and in place of the container 500 there may be provided an aseptic connector, a vacutainer or the like. Any combination of these containers is contemplated herein.

1. An apparatus suitable for use in handling biological material, for directly introducing or removing material to, or from, a container, comprising:
a body comprising at least one resealable port therein; and
a cover comprising an aperture and wherein the cover is disposed over an upper side of the body, and wherein the cover is moveable, the aperture of the cover is configured to expose at least part of the resealable port, when the aperture is disposed over the resealable port.

2. An apparatus according to claim 1, wherein the body comprises an upper surface and a lower surface, the cover is disposed over the upper surface, and the at least one resealable port extending through the body from the upper surface to the lower surface.

3. An apparatus according to claim 2, wherein the body comprises a plurality of longitudinal passageways extending from the upper surface to a recess formed in the lower surface, each longitudinal passageway comprising a resealable port and an annular recess comprising an annular resealable port.

4. An apparatus according to claim 3, wherein each resealable port in each longitudinal passageway and the annular resealable port are integrally formed.

5. An apparatus according to claim 1, wherein the at least one resealable port is a hermetic seal.

6. An apparatus according to claim 5, wherein the hermetic seal is a septum seal.

7. The apparatus according to claim 6, wherein the septum seal and the body are co-molded.

8. The apparatus according to claim 1, the cover further comprising a connector mechanism for connecting to a portion of a container, a bioreactor or a connector.

9. The apparatus according to claim 8, wherein the connector mechanism comprises a threaded portion configured to engage with a corresponding threaded portion of a container, a bioreactor or a connector.

10. The apparatus according to claim 1, wherein the cover comprises a centrally disposed opening at least partly surrounded by an upstanding wall.

11. The apparatus according to claim 10, wherein the body comprises a central hub having a coupling element, the central hub extending through the centrally disposed opening and the coupling element operably coupled to the upstanding wall.

12. The apparatus according to claim 1, wherein the cover comprises a circumferential skirt that is configured and arranged to removably attach to a circumferential rim of the body.

13. The apparatus according to claim 1, wherein the cover comprises a substantially circumferential rail that is configured and arranged to be received within a substantially circumferential groove of the body.

14. An apparatus according to claim 1, wherein the cover and the body are rotatable with respect to one another.

15. An apparatus according to claim 1, wherein a lower surface of the body includes a connector mechanism for connecting to a portion of a container, a bioreactor or a connector.

16. An apparatus according to claim 15, wherein the connector mechanism is a threaded portion configured to engage with a corresponding threaded portion of a container, a bioreactor or a connector, the threaded portion comprising an anti-rotational element configured and arranged to resist rotation of a container, a bioreactor or a connector, in use.

17. An apparatus according to claim 16, wherein the anti-rotational element comprises one or more trapezoidal threads of the threaded portion.

18. An apparatus according to claim 1, further comprising a detachable cover disposed over at least a portion of the at least one resealable port.

19. A system suitable for use in handling biological material comprising:
an apparatus suitable for use in handling biological material, for directly introducing or removing material to, or from, a container, the apparatus comprising:
a body comprising at least one resealable port therein; and
a cover comprising an aperture and wherein the cover is disposed over an upper side of the body, and wherein the cover is moveable, the aperture of the cover is configured to expose at least part of the resealable port, when the aperture is disposed over the resealable port.
; and
a container, a bioreactor and/or a connector sealingly engaged with the at least one resealable port.

20. A method of operating an apparatus suitable for use in handling biological material, for directly introducing or removing material to, or from, a container, comprising:
providing an apparatus suitable for use in handling biological material, for directly introducing or removing material to, or from, a container, the apparatus comprising:
a body comprising at least one resealable port therein; and
a cover comprising an aperture and wherein the cover is disposed over an upper side of the body, and wherein the cover is moveable, the aperture of the cover is configured to expose at least part of the resealable port, when the aperture is disposed over the resealable port;
sealingly engaging a first container to the at least one resealable port at a lower surface of the body;
sealingly engaging a second container to the at least one resealable port at an upper surface of the body;
providing fluid communication between the container and the bioreactor by providing a fluid passageway through the at least one resealable port; and
introducing material from the second container into the first container, or removing material from the first container into the second container.
